(19) United States
(12) Patent Application Publication
(10) Pub. No.: US 2021/0047623 A1
(43) Pub. Date: Feb. 18, 2021

(54) CRISPR KNOCK-OUT OF THE ALPHA-SYNUCLEIN TRIPLICATION MODEL OF PARKINSON'S DISEASE

(71) Applicant: PARKINSON'S INSTITUTE, Mountain View, CA (US)

(72) Inventor: Birgitt SCHÜLE, Mountain View, CA (US)

(21) Appl. No.: 16/965,573

(22) PCT Filed: Jan. 29, 2019
(86) PCT No.: PCT/US2019/015714

§ 371 Date: Jul. 28, 2020

Related U.S. Application Data
(60) Provisional application No. 62/624,052, filed on Jan. 30, 2018.

(51) Int. Cl. C12N 5/074; C12N 9/22; C12N 15/113; C12N 5/0797

(52) U.S. Cl. CPC C12N 5/0696; C12N 9/22; C12N 15/113; C12N 2510/00; C12N 2310/20; C12N 2800/80; C12N 5/0623

(57) ABSTRACT

Human-derived isogenic induced pluripotent stem cells (iPSCs) with copy number variation for alpha-synuclein, and methods of use thereof, are provided.

### CROSS REFERENCE

This application claims the benefit of U.S. Provisional Application No. 62/624,052 filed Jan. 30, 2018, which is incorporated herein by reference in its entirety.

### BACKGROUND OF THE INVENTION

Complex diseases such as Parkinson's disease (PD), as well as many others, may involve genetic variations such as mutations and/or copy number variations (CNV) or may be idiopathic or sporadic. The accumulation and aggregation α-synuclein protein (α-syn) is a critical event in PD pathophysiology, impairing neuronal function and contributing to dopaminergic neuronal cell death. The pathogenic genomic triplication of the alpha-synuclein (SNCA) gene in patients results in early onset rapidly progressive parkinsonism with diffuse Lewy body pathology and severe autonomic involvement, directly linking increased gene expression of wild-type α-syn and disease development.

### SUMMARY OF THE INVENTION

Disclosed herein, are isogenic induced pluripotent cell line produced from induced pluripotent stem cell with alpha-synuclein (SNCA) gene triplication, produced by: (a) contacting the stem cell with SNCA gene triplication with (i) a synthetic polynucleotide that targets a target sequence in one or more of the SNCA genes, and (ii) a genetically engineered vector comprising a gene which encodes a nucleic acid-guided nuclease; and (b) assessing the cell for copies of the SNCA gene. The induced pluripotent stem cell with alpha-synuclein (SNCA) gene triplication may be human-derived. One or more of the SNCA genes may be a functional SNCA gene. The synthetic polynucleotide may be a guide nucleic acid. The guide nucleic acid may be a guide RNA (gRNA). The target sequence may be in exon 2 of the one or more SNCA genes. The target sequence may be in exon 3 of the one or more SNCA genes. The target sequence may be in exon 4 of the one or more SNCA genes. The target sequence may be in exon 5 of the one or more SNCA genes. The target sequence may comprise: 5′ GAGAAAACCAAACAGGGTG 3′, 5′ GGACTTTCAAAGGCCAAGG 3′, 5′ GCTGCTGAGAAAACCAAAC 3′, 5′ GCTTCTGCCACACCCTGTT 3′, or 5′ GCAGCCACAACTCCCTCCT 3′. The nuclease may introduce a double strand break in the target sequence in one or more the SNCA genes. The target sequence in the one or more SNCA genes may be modified by non-homologous end joining. The nucleic acid-guided nuclease may be a CRISPR nuclease. The CRISPR nuclease may be Cas9. The cell may have three copies of a functional SNCA gene. The cell may have two copies of a functional SNCA gene. The cell may have one copy of a functional SNCA gene. The cell may have zero copies of a functional SNCA gene. The cell may have normal karyotype. The cell may maintain expression of pluripotency markers. The cell may maintain differentiation potential. SNCA mRNA expression in the cell may be proportional to the copies of functional SNCA genes in the cell. The cell may be present in a cell culture.

Disclosed herein, are isogenic induced pluripotent cell line comprising three copies of alpha-synuclein (SNCA) gene, wherein the cell line may be produced from induced pluripotent stem cell with alpha-synuclein (SNCA) gene triplication. The induced pluripotent stem cell with alpha-synuclein (SNCA) gene triplication may be human-derived. The SNCA gene may be functional SNCA gene. The cell may have normal karyotype. The cell may maintain expression of pluripotency markers. The cell may maintain differentiation potential. SNCA mRNA expression in the cell is increased compared to SNCA mRNA expression in a control cell. SNCA mRNA expression in the cell may be comparable to SNCA mRNA expression in a control cell. The control cell may comprise two copies of wild-type SNCA gene. The cell may be present in a cell culture.

Disclosed herein, are isogenic induced pluripotent cell line comprising two copies of alpha-synuclein (SNCA) gene, wherein the cell line may be produced from induced pluripotent stem cell with alpha-synuclein (SNCA) gene triplication. The induced pluripotent stem cell with alpha-synuclein (SNCA) gene triplication may be human-derived. The SNCA gene may be functional SNCA gene. The cell may have normal karyotype. The cell may maintain expression of pluripotency markers. The cell may maintain differentiation potential. SNCA mRNA expression in the cell may be increased compared to SNCA mRNA expression in a control cell. SNCA mRNA expression in the cell may be comparable to SNCA mRNA expression in a control cell. The control cell may comprise two copies of wild-type SNCA gene. The cell may be present in a cell culture.

Disclosed herein, are isogenic induced pluripotent cell line comprising one copy of alpha-synuclein (SNCA) gene, wherein the cell line may be produced from induced pluripotent stem cell with alpha-synuclein (SNCA) gene triplication. The induced pluripotent stem cell with alpha-synuclein (SNCA) gene triplication may be human-derived. The SNCA gene may be functional SNCA gene. The cell may have normal karyotype. The cell may maintain expression of pluripotency markers. The cell may maintain differentiation potential. SNCA mRNA expression in the cell may be comparable to SNCA mRNA expression in a control cell. SNCA mRNA expression in the cell may be decreased compared to SNCA mRNA expression in a control cell. The control cell may comprise two copies of wild-type SNCA gene. The cell may be present in a cell culture.

Disclosed herein, are isogenic induced pluripotent cell line comprising zero copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) triplication may be human-derived. The zero copies may be zero functional copies of SNCA gene. The cell may have normal karyotype. The cell may maintain expression of pluripotency markers. The cell may maintain differentiation potential. The cell may have reduced differentiation potential compared to a control cell. SNCA mRNA expression in the cell may be decreased compared to SNCA mRNA expression in a control cell. The control cell may comprise two copies of wild-type SNCA gene. The cell may be present in a cell culture.

Disclosed herein, are neuronal precursor cell line derived from an isogenic induced pluripotent cell line disclosed herein. Disclosed herein, are neuronal cell line derived from an isogenic induced pluripotent cell line disclosed herein. The derived cell line may be used to derive a dopaminergic (DA) neuron.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C illustrate analysis of mitochondria respiratory chain complexes in neuroprecursor cells (NPCs). FIG. 1A illustrates a Western analysis of mitochondria respiratory chain complex IV, subunit I (C IV) expression in NPCs derived from Control (Ctrl) and SNCA-Triplication (SNCA-Tri) iPSCs. Heat shock protein 90 (HSP90) was used as loading control. Analysis of mitochondrial respiratory Complex I protein content (FIG. 1B) and Complex IV protein content and activity (FIG. 1C) in NPCs from Control (Ctrl), a-synuclein triplication (SNCA-Tri) under control conditions (HG), and after challenge with Rotenone (HG+Rot) or nutrient withdrawal (NG) determined by native ELISA based assays. Data from 2 independent experiments with 2 replicated each are shown (+/−SD, *p≤0.01, **p≤0.001; ANOVA).

FIG. 2 illustrates the GeneArt Genomic Cleavage Detection results for HEK293 cells transfected with pGS-U6-gRNA and pGS-CMV-hCas9 plasmids. Top shows gel electrophoresis of enzyme digested DNA fragments. Bottom shows the comparison of indel mutation % values from TIDE analysis and GeneArt Genomic Cleavage Kit.

FIG. 3A-FIG. 3B illustrate a methodology used to target SNCA locus. FIG. 3A illustrates the SNCA genomic locus of Chr.4q22.1 and CRISPR targeted gene region of the SNCA gene exon 2. FIG. 3B illustrates a novel concept for SNCA gene knockout iPSC model. Introduce sequentially guided by CRISPR frame-shift mutations via non-homologous end-joining in the first coding exon of the SNCA gene to generate an ‘SNCA gene dosage’ model at its endogenous locus.

FIG. 4 illustrates Cel-1 assay used to measure reagent (sgRNA) cutting efficiency in HEK293T. HEK293 were transfected with each reagent to assess cutting at the target locus via Cel-1 assay. All five reagents showed cutting efficiency at 10-13%. Lanes 1-5 represent reagents 1 through 5 and lane 6 represents the uncut control.

FIG. 5 illustrates cells were transfected with CRISPR3 (highest cutting reagent). In order to increase the chances of getting all four alleles cut, transfection with the nucleases was performed 3 times sequentially over 6 weeks of time period. Cel-1 assay showing increased cutting efficiency after three consecutive transfections on pooled human iPSCs with SNCA genomic triplication. Lanes 1, 2 and 3 represent cutting with CRISPR3 after each round, with cutting levels reaching 24.5% after the third round. Lanes 4 and 5 represent cutting with another pulse of gRNA 24 hours after the initial transfection. Cutting efficiency reaches 35% with the additional pulse of gRNA. Lane 7 represents the uncut control cells.

FIG. 6 illustrates SNCA expression in isogenic clones by determining SNCA mRNA expression in iPSCs. CRISPR knock-out (KO) clones are compared to normal control and parental SNCA triplication iPSC clones. Isogenic lines (1^{st} set) show mRNA reduction in proportion to number of KO copies. However, when compared to the parental SNCA triplication, the KO lines express proportionally higher levels of a-syn. There is still residual expression in the SNCA 4KO. Two other 2KO clones (2^{nd} set) show similar SNCA expression as control.

FIG. 7 illustrates a schematic for timeline for neural induction of edited iPSCs and their differentiation into mature neurons. Media composition and added supplements (suppl.) are abbreviated as follows: SB43: SB431542; Dor: Dorsomorphin; Putr: Putrescin; Transf.: Transferrin; Na-Sel.: Na-Selenite; Ins.: Insulin.

FIG. 8 illustrates a schematic for differentiation of SNCA isogenic iPSC clones into dopaminergic (DA) neurons to investigate phenotypic differences among lines.

FIG. 9 illustrates neuronal differentiation protocol exhibits homogenous population of floorplate marker forkhead box A2 (FOXA2). Upper panel exhibits pluripotent morphology of iPSCs. Lower panel exhibits morphology of cells after 10 days treated with specification media.

FIG. 10 illustrates FPp0 at 24 hours after passaging from day10. Cells uniformly express midbrain marker FOXA2 and neuro-precursor marker, NESTIN. Counterstained with DAPI at 10× magnification.

FIG. 11 illustrates expression of FOXA2 and tyrosine hydroxylase (TH) in DA neurons after 35 days of differentiation. DA neurons are still highly positive FOXA2. Co-localization of FOXA2 and TH confirms FOXA2 expression is critical to develop DA neurons. In 2F4_4KO line, even though high expression of FOXA2 is noticed, but TH expression is very limited. Cells were counterstained with DAPI at 10× magnification.

FIG. 12 illustrates mature day35 DA neurons stained with TUJ1 for neurons over total cell number. Cells were also double stained with TH for DA neurons. TH positive cells were co-localized with TUJ1 stained cells. Higher density of DA neurons is observed in control, compared to triplication and all isogenic lines. However, among all isogenic lines, DA neurons of 2KO line view morphological and quantity similarity with control. Cells were counterstained with DAPI at 10× magnification.

FIG. 13A-FIG. 13B illustrate SNCA and TH expression analysis for different time-points during neuronal differentiation. SNCA and TH mRNA expression at day0 (iPSCs), day10 (FPp0) and day35 (mature neurons) determined by Taqman qPCR. FIG. 13A illustrates SNCA expression is almost 60-70 fold higher at day35 compared to day0 or day10. FIG. 13B illustrates TH expression is 100 to 2000 fold higher compared to day0 and day10. At day 35, higher TH noted in 1KO, 2KO, and 3KO lines compared to SNCA triplication line.

FIG. 14A-FIG. 14D illustrate transcriptome analysis of the isogenic iPSC lines. FIG. 14A is a statistic chart of differentially expressed genes of each pair. Only >2-fold changes are presented. The first two columns of the graph compare the parental iPSC lines to the CRISPR knockout line with 2 frameshift mutations (2KO). 401 genes were downregulated, and 411 genes were upregulated. The middle two columns compare the parental iPSC lines to the CRISPR knockout line with 4 frameshift mutations (4KO). 156 were upregulated and 231 genes were downregulated. The last two columns compare the parental iPSC lines to the CRISPR knockout line with 3 frameshift mutations (3KO). 443 were upregulated and 807 genes were downregulated. FIG. 14B is gene ontology analysis of differentially expressed genes. CRISPR knockout line (1F6) with 2 frameshift mutations (2KO) is compared to the parental control. Several biological processes, cellular components, and molecular function have been identified to be affected due to a 2-fold change in SNCA expression. FIG. 14C is pathway enrichment analysis of differentially expressed genes. The left panel shows the top 20 pathways enriched in CRISPR knockout line with 2 frameshift mutations (2KO) compared to parental control. The signaling pathways are: signaling pathways regulating pluripotency genes, Ras signaling pathway, proteoglycans in cancer, protein digestion and absorption, PI3K/AKT signaling pathway, p53 signaling pathway, osteoclast differentiation, notch signaling pathway, mineral absorption, microRNAs in cancer, MAPK signaling pathway, Linoleic acid metabolism, FoxO signaling pathway, ether lipid metabolism, ECM-receptor interaction, choline metabolism in cancer, cell adhesion molecules, axon guidance, arachidonic acid and metabolism, alpha-linolenic acid metabolism. The left panel shows the top 20 pathways enriched in CRISPR knockout line with 4 frameshift mutations (2KO) compared to parental control. The signaling pathways are: Wnt signaling pathway, toxoplasmosis, Toll-like receptor signaling pathway, signaling pathways regulating pluripotency genes, rheumatoid arthritis, proteoglycans in cancer, pertussis, p53 signaling pathway, NF-kappa B signaling pathway, microRNAs in cancer, Leishmaniasis, Legionellosis, HTLV-1 infection, Hedgehog signaling pathway, FoxO signaling pathway, complement and coagulation cascades, Chagas disease, cell adhesion molecules, axon guidance. FIG. 14D is a prediction and annotation of novel transcripts. Each column presents an iPSC clone: the first column is CRISPR knockout line (1F6) with 2 frameshift mutations (2KO), the second column is CRISPR knockout line (2F4) with 4 frameshift mutations (4KO), the third column is CRISPR knockout line (4F6) with 3 frameshift mutations (3KO), the last column is the parental control (HUF4).

### DETAILED DESCRIPTION OF THE INVENTION

### Overview

Disclosed herein, iPSC technology is combined with gene editing to establish isogenic cellular tools which express varying SNCA gene copy numbers. CRISPR tools were generated to introduce double-strand breaks in the first coding exon of SNCA gene. Human iPSCs from a SNCA triplication carrier were growth adapted to single cell cloning and were transfected with the CRISPR constructs several rounds before genotyping of individual clones. Clones with different mutant alleles relating to 4 knockout (KO), 3KO, 2KO, 1KO were generated.

Alpha-synuclein (α-syn) is critical for normal function of subcellular membrane systems such as mitochondria and mitochondria-associated membranes, and may be involved in signal transduction, membrane remodeling, stabilization, and function of membrane-associated proteins. The accumulation and aggregation α-synuclein protein (α-syn) is a critical event in Parkinson's disease (PD) pathophysiology, impairing neuronal function and contributing to dopaminergic neuronal cell death. The pathogenic genomic triplication of the alpha-synuclein (SNCA) gene (chromosomal locus 4q21, size 1.7 Mb) in patients results in early onset rapidly progressive parkinsonism with diffuse Lewy body pathology and severe autonomic involvement, suggesting a direct link between increased gene expression of wild-type α-syn and disease development. Overexpression of α-syn as is related to the SNCA genomic triplication is linked to increased susceptibility for oxidative stress and impairment of neuronal maturation in patient-derived fibroblasts or induced pluripotent stem cell (iPSC) models.

Currently, there is no cure, early detection mechanism, preventative treatment, or effective way to slow the disease progression. A model that replicates the fundamental features of the disease at the genomic level is needed. Induced pluripotent stem cells (iPSCs), developed from patients presenting with genetic and complex diseases, further differentiated into specific cell types, known to be affected in the natural progression of disease, will provide heretofore unavailable cellular tools for identification of disease mechanisms.

The gene dosage of α-syn protein in cell lines disclosed herein is differentially regulated by expression of different α-syn (SNCA) gene copies in their genome and may be used to determine the role of altered α-syn levels on membrane associated processes and general cell function. This set of new cell lines may be used to determine the physiological and detrimental effects of varying α-syn levels, thus greatly simplifying the experimental paradigm that arises when overexpressing proteins or downregulating gene expression, i.e. RNA interference. Dopaminergic neurons derived from these isogenic lines are analyzed for viability, differentiation potential and morphological as well as physiological changes to evaluate the effect of different ‘gene doses’ of alpha-synuclein.

Disclosed herein, are unique in vitro model systems to study the impact of α-syn in an isogenic background. This system may be extremely useful for the study of α-syn associated pathways. The novel in vitro model systems disclosed herein have two major advantages over animal models and other in vitro models. First, these models enable one to work in the human system. Second, CRISPR gene editing technology is used to create isogenic cell lines. These newly engineered iPSC lines represent near perfect controls as they literally differ only by the introduced genetic modification. This minimizes the challenges of biological noise and variability. Overall, the sequential targeting of the SNCA locus is an innovative approach to study overexpression and reduced expression of α-syn. This is critical as α-syn downregulation is considered a therapeutic strategy in PD.

Isogenic Induced Pluripotent Cell (iPSC) Line with Different Functional Copy Numbers of the SNCA Gene

Disclosed herein, are isogenic iPSC lines produced from induced pluripotent stem cell with alpha-synuclein (SNCA) gene triplication. The isogenic iPSC line may have three copies of functional SNCA gene. The isogenic iPSC line may have two copies of functional SNCA gene. The isogenic iPSC line may have one copy of functional SNCA gene. The isogenic iPSC line may have zero copy of functional SNCA gene.

iPSC Line with Three Copies of Functional SNCA Gene

Disclosed herein, is isogenic iPSC line comprising three copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be increased compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The cell may be present in a cell culture. The iPSC line with three copies of functional SNCA gene may be used to derive a neuronal precursor cell line. The iPSC line with three copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with Two Copies of Functional SNCA Gene

Disclosed herein, is isogenic iPSC line comprising two copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be higher compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The cell may be present in a cell culture. The iPSC line with two copies of functional SNCA gene may be used to derive a neuronal precursor cell line. The iPSC line with two copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with One Copy of Functional SNCA Gene

Disclosed herein, is isogenic iPSC line comprising one copy of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The cell may have reduced differentiation potential compared to a control cell, wherein the control cell comprises two copies of wild-type SNCA gene. The morphology of the cell during initial specification may be comparable to a control cell comprising two copies of the wild-type SNCA gene. The SNCA mRNA expression in the cell may be higher compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The SNCA mRNA expression in the cell may be decreased compared to SNCA mRNA expression in a control cell wherein the control cell comprises two copies of wild-type SNCA gene. The SNCA mRNA expression in the cell may be decreased by about 50% compared to SNCA mRNA expression in a control cell wherein the control cell comprises two copies of wild-type SNCA gene. The cell may be present in a cell culture. The iPSC line with one copy of functional SNCA gene may be used to derive a neuronal precursor cell line. The iPSC line with one copy of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with Zero Copies of Functional SNCA Gene

Disclosed herein, is isogenic iPSC line comprising zero copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may not maintain expression of pluripotency markers. The cell may maintain differentiation potential. The cell may have reduced differentiation potential compared to a control cell, wherein the control cell comprises two copies of wild-type SNCA gene. The cell may have almost no differentiation potential compared to a control cell, wherein the control cell comprises two copies of wild-type SNCA gene. The cell may have no differentiation potential compared to a control cell, wherein the control cell comprises two copies of wild-type SNCA gene. The morphology of the cell during initial specification may be comparable to a control cell comprising two copies of the wild-type SNCA gene. The SNCA mRNA expression in the cell may be decreased compared to SNCA mRNA expression in a control cell wherein the control cell comprises two copies of wild-type SNCA gene. The cell may have almost no SNCA mRNA expression compared to SNCA mRNA expression in a control cell wherein the control cell comprises two copies of wild-type SNCA gene. The cell may have almost no SNCA mRNA expression. The cell may have no SNCA mRNA expression. The cell may be present in a cell culture. The iPSC line with zero copies of functional SNCA gene may be used to derive a neuronal precursor cell line. The iPSC line with zero copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

Method of Generating iPSC Lines with Different Functional Copy Numbers of the SNCA Gene

Disclosed herein, are isogenic iPSC lines produced from induced pluripotent stem cell with alpha-synuclein (SNCA) gene triplication. The isogenic iPSC lines may be produced by: (a) contacting the induced pluripotent stem cell with SNCA gene triplication with (i) a synthetic polynucleotide that targets a target sequence in one or more of the SNCA genes, and (ii) a genetically engineered vector comprising a gene which encodes a nucleic acid-guided nuclease; and (b) assessing the cell for copies of the SNCA gene. The induced pluripotent stem cell with SNCA gene triplication may be contacted with one or more synthetic polynucleotides that target a target sequence in one or more of the SNCA genes. The induced pluripotent stem cell with alpha-synuclein (SNCA) gene triplication may be human-derived. The one or more of the SNCA genes may be a functional SNCA gene. The one or more of the SNCA genes may be a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The target sequence may be in exon 2 of the one or more SNCA genes. The target sequence may be in exon 3 of the one or more SNCA genes. The target sequence may be in exon 4 of the one or more SNCA genes. The target sequence may be in exon 5 of the one or more SNCA genes. The target sequence may comprise: 5′ GAGAAAACCAAACAGGGTG 3′, 5′ GGACTTTCAAAGGCCAAGG 3′, 5′ GCTGCTGAGAAAACCAAAC 3′, 5′ GCTTCTGCCACACCCTGTT 3′, or 5′ GCAGCCACAACTCCCTCCT 3′. The nuclease may introduce a double strand break in the target sequence in one or more the SNCA genes. The target sequence in the one or more SNCA genes may be modified by non-homologous end joining. The nucleic acid-guided nuclease may be a CRISPR nuclease. The CRISPR nuclease may be Cas9. The CRISPR nuclease may be Cpf1. The guide nucleic acid may be guide DNA and the target may be modified by Argonaute proteins. The cell may have three copies of a functional SNCA gene. The cell may have two copies of a functional SNCA gene. The cell may have one copy of a functional SNCA gene. The cell may have zero copies of a functional SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be proportional to the copies of functional SNCA genes in the cell. The cell may be present in a cell culture. The isogenic iPSC line may be used as cellular tool for in vitro studies to understand the molecular mechanism of α-syn under expression and overexpression in the pathogenesis of PD.

### Targetable Nucleic Acid Cleavage Systems

Methods disclosed herein comprise targeting cleavage of specific nucleic acid sequences using a site-specific, targetable, and/or engineered nuclease or nuclease system. Such nucleases may create double-stranded break (DSBs) at desired locations in a genome or nucleic acid molecule. In other examples, a nuclease may create a single strand break. In some cases, two nucleases are used, each of which generates a single strand break.

The one or more double or single strand break may be repaired by natural processes of homologous recombination (HR) and non-homologous end-joining (NHEJ) using the cell's endogenous machinery. Additionally or alternatively, endogenous or heterologous recombination machinery may be used to repair the induced break or breaks.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length.

A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed within a cell. A CRISPR nuclease may be encoded on a chromosome, extrachromosomally, or on a plasmid, synthetic chromosome, or artificial chromosome. A CRISPR nuclease may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA within a cell. In some examples, gRNAs may be provided or delivered to a cell expressing a CRISPR nuclease. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

A CRISPR system may be a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed within a cell. Argonaute may be encoded on a chromosome, extrachromosomally, or on a plasmid, synthetic chromosome, or artificial chromosome. Additionally or alternatively, an Argonaute protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

Guide DNAs may be provided by genetic or episomal DNA within a cell. In some examples, gDNA are reverse transcribed from RNA or mRNA within a cell. In some examples, gDNAs may be provided or delivered to a cell expressing an Ago protein. Guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed within a cell. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked, prior to delivery to a cell. A nuclease fusion protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

### Guide Nucleic Acid

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid that is compatible with the nucleic acid-guided nucleases.

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

### EXAMPLES

### Example 1

### Generation of Isogenic iPSC Lines with Different Wild-Type Copies of the SNCA Gene

Multiple clonal iPSC lines from skin cells of a PD patient carrying a triplication of the SNCA gene have been reprogrammed using a retroviral system with four factors encoding OCT4, KLF4, SOX2, and cMYC9. All iPSC lines were characterized for pluripotency, differentiation potential, silencing of transgenes, and have a normal karyotype.

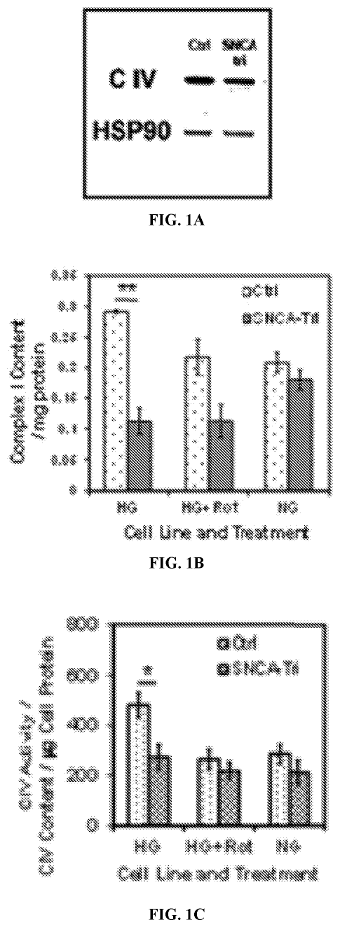

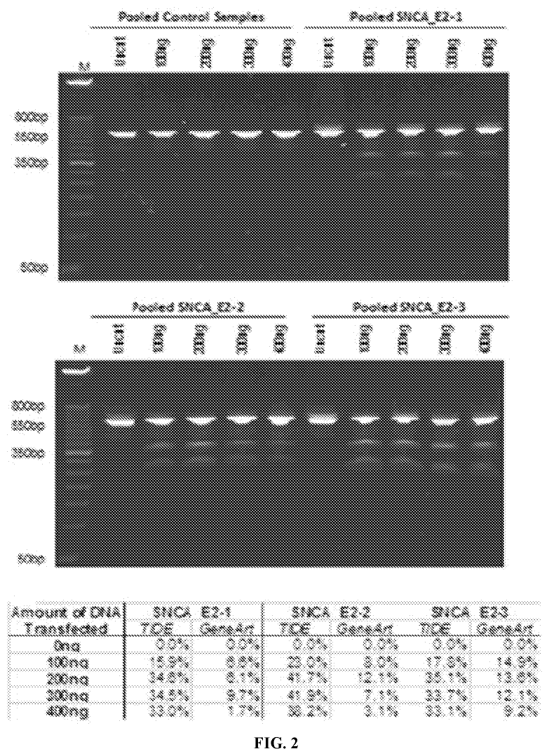

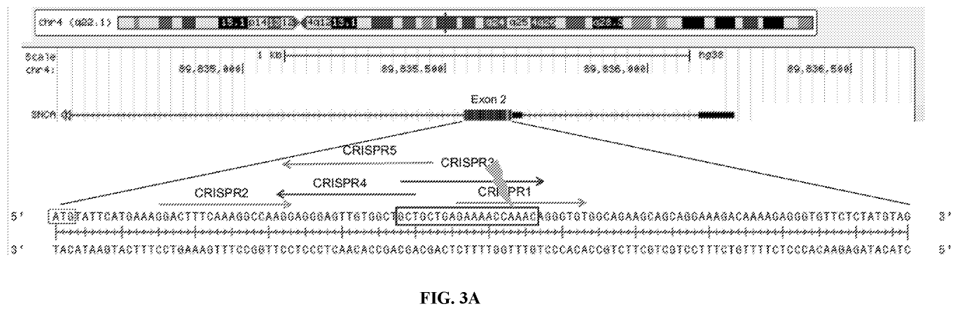

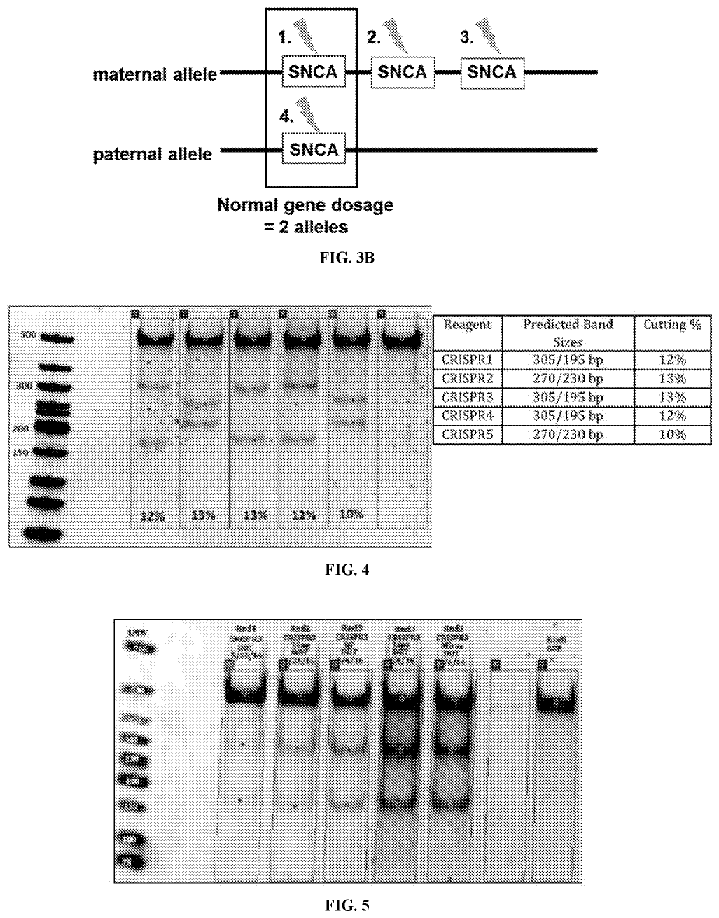

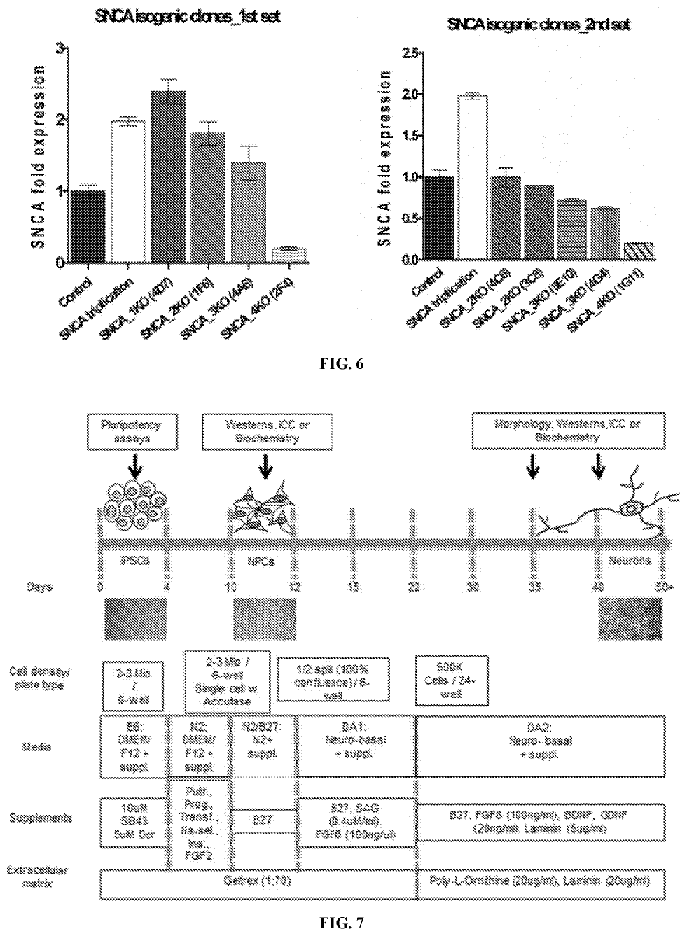

The function of mitochondrial respiratory chain, and particularly mitochondrial Complex I has been shown to be affected in PD, and fibroblasts with the SNCA gene triplication have altered mitochondrial complex I function. Preliminary results also show that these defects are present in there from derived NPCs, which also presented with reduced complex IV protein levels and reduced complex IV function (FIG. 1A-FIG. 1C). In addition, SNCA-triplication cell lines exhibit reduced mitochondrial protein import function.

### Example 2

### Generation of Isogenic iPSC Lines with Different Functional Copy Numbers of the SNCA Gene

iPSC clones from a donor with an SNCA gene triplication (descendant from the Iowa kindred) were used to engineer and genetically characterize a panel of patient-derived isogenic induced pluripotent stem cells lines that carry different functional copies of the SNCA gene, ranging from four to zero functional gene copies). The set of iPSC lines may be used as a model system for further functional studies of the physiological role of α-syn in human neurons.

Induced pluripotent stem cell (iPSC) culture and maintenance: iPSCs were cultured on Geltrex with manual passaging every 6-7 days. Essential 8 media was changed daily.

CRISPR reagent design-build, transfection and screening: Cells were adapted to single-cell passaging techniques required for efficient gene editing. Several small guide (sg) RNAs that specifically target the SNCA gene exons 2 through 5 were experimentally determined and their target specificity in HEK293 cells was validated (FIG. 2). One sgRNA with high cutting efficiency (SNCA_E2_2, about 40%) was identified. Five gRNAs (reagents) were designed and built to exon 2 of SNCA (Table 1). HEK293T were transfected with each reagent to assess cutting at the target locus via Cel-1 assay. Transfection with the nucleases in iPSCs was performed 3 times sequentially (over 6 weeks).

TABLE 1
A panel of tested CRISPR guide RNAs
used to knock-out SNCA.

| sgRNA ID | Target Sequence |
|---|---|
| CRISPR1 | 5′ GAGAAAACCAAACAGGGTG 3′ |
| CRISPR2 | 5′ GGACTTTCAAAGGCCAAGG 3′ |
| CRISPR3 | 5′ GCTGCTGAGAAAACCAAAC 3′ |
| CRISPR4 | 5′ GCTTCTGCCACACCCTGTT 3′ |
| CRISPR5 | 5′ GCAGCCACAACTCCCTCCT 3′ |

Transfected pools were screened for the presence of the indels indicating repair via NHEJ at the cut-sites. Cel-1 assay was performed to assess the level of cutting at the sites after each round of transfection. The consolidated clones were initially assessed for knockout alleles utilizing droplet digital PCR. The sequence confirmed clones were further expanded to make the final cell banks for future experiment.

TABLE 2
CRISPR-modified SNCA alleles in human iPSC

| Clone # | KO Status | Allele Status | Notes |
|---|---|---|---|
| 1 | 4 KO | del4/del5 (no wt) | Internal clone 1G11; no evidence of WT in 55 sequences |
| 2 | 4 KO | del5/del26/del8/ins14 | Internal clone 2F4; see 4 KO alleles |
| 3 | 4 KO | del2/del4/del5 (no wt) | Internal clone 3B2; no evidence of WT in 50 sequences |
| 4 | 3 KO | wt/ins1/del5/ins8 | Internal clone 4A6; see all 4 alleles |
| 5 | 3 KO | wt/del5/del19/ins1 | Internal clone 4G4; see all 4 alleles |
| 6 | 3 KO | wt/del5/del19/ins13 | Internal clone 5E10; se all 4 alleles |
| 7 | 2 KO | wt/del5/del17 | Internal clone 1F6; see 3 alleles (assumes 2 WT; |
| | | | matches ddPCR) |
| 8 | 2 KO | wt/del4/del5 | Internal clone 3C8; see 3 alleles (assumes 2 WT; matches |
| | | | ddPCR) |
| 9 | 2 KO | wt/del4/del34 | Internal clone 4C6; see 3 alleles (assumes 2 WT; matches |
| | | | ddPCR) |
| 10 | n/a | wt/del5/del68/del34/del2 | Internal clone 4B6; NOT EXPANDED; apparent mixed |
| | | | clone |
| 11 | 1 KO | wt/del5 | Internal clone 4D7; see 2 alleles (assumes 3 WT; |
| | | | matches ddPCR) |

Clonally expanded gene-edited iPSC lines are screened with PCR-based heteroduplex-detecting assays. Alternatively, gene-edited iPSCs clones are screened using hetero-duplex analysis of PCR-amplified target sites by denaturing high-performance liquid chromatography (DHPLC), e.g. Transgenomics® WAVE system or bioinformatics approach using Sanger sequence information to detect small insertions or deletions by decomposition analysis (TIDE). Clones that show targeted disruption of the SNCA gene in the initial screen are selected and further characterized by the following strategies: 1) By cloning of SNCA target location-spanning PCR fragments and Sanger sequencing of individual clones; 2) By quantitative RT-PCR analysis of SNCA gene transcripts, to detect differential expression levels of functional SNCA mRNAs; 3) By analyzing α-syn protein levels by immunoblotting.

All positive clones with the desired functional copy number of the SNCA gene (zero copies/knock-out, 1, 2, 3, and 4 functional copies) are characterized for maintenance of pluripotency, differentiation potential, and normal karyotype.

If the targeting with a single gRNA does not result in disturbance of all alleles in an iPSC clone, either cumulative or sequentially sgRNAs targeting to other exons of the SNCA gene is employed. Additional sgRNAs to exons 3 to 5 are designed and cloned.

### Example 3

### Testing the Effect of α-syn on Structure and iPSC-Differentiated Neurons with Different Functional Gene Copies of the SNCA Gene

A panel of assays has been developed to characterize mitochondrial function and bioenergetics and is applied to the derived SNCA CRISPR/Cas9-derived iPS neuronal cultures. The effect of different SNCA functional copies on steady state levels and dynamics of subcellular membrane systems (mitochondria, lysosomes, endoplasmic reticulum (ER), and particular the mitochondrial respiratory chain assembly and function is determined. α-syn protein modification and changes in level of α-syn interacting proteins is also studied.

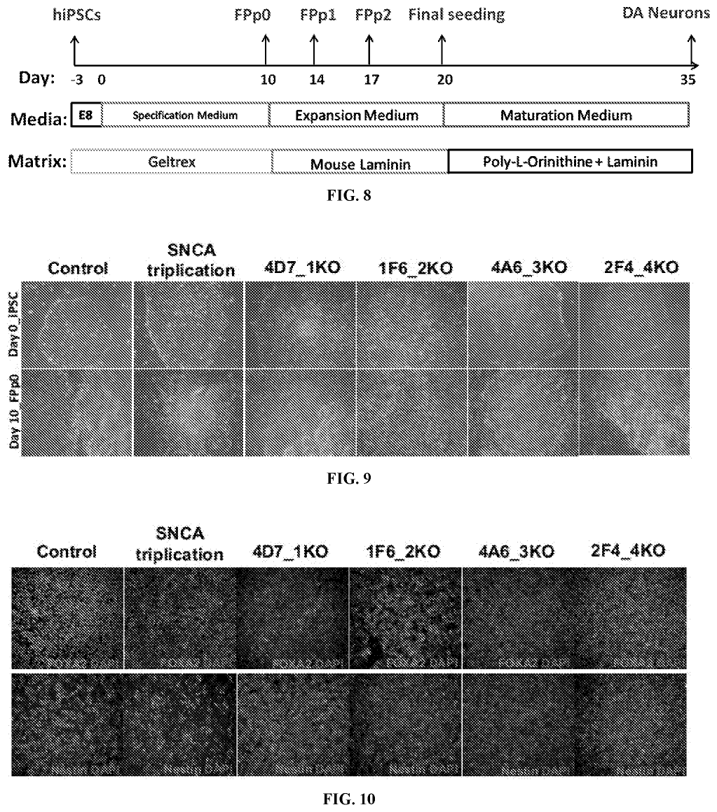

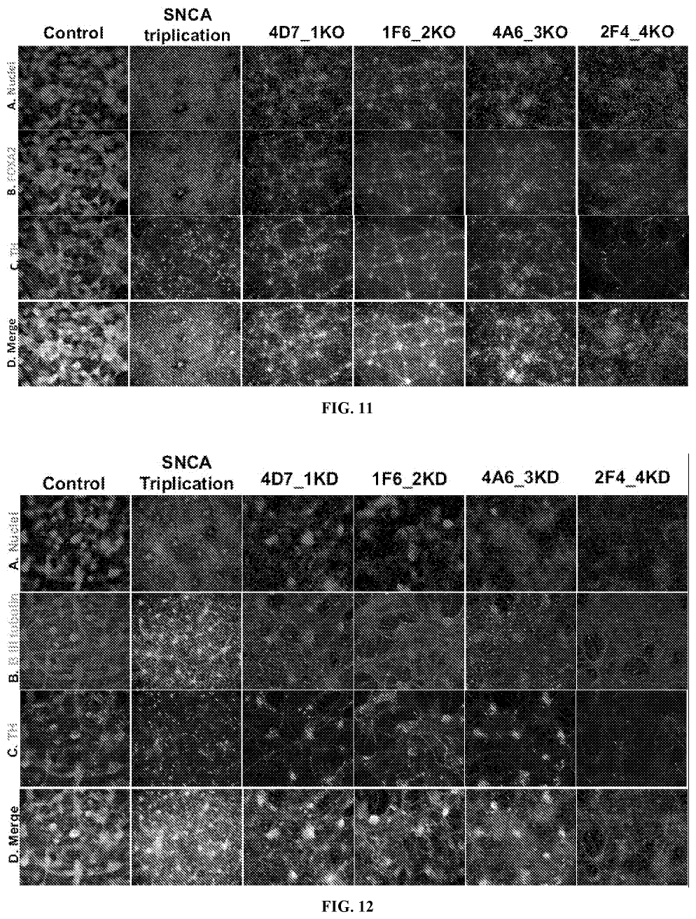

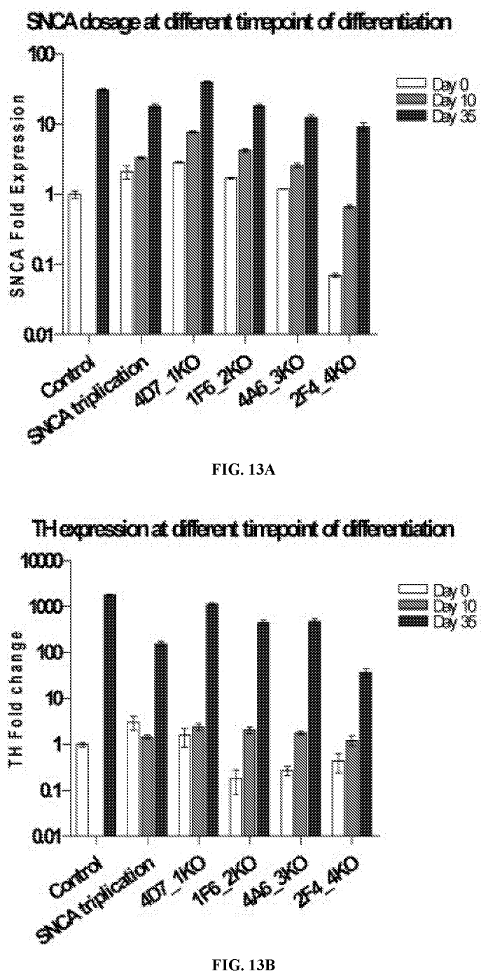

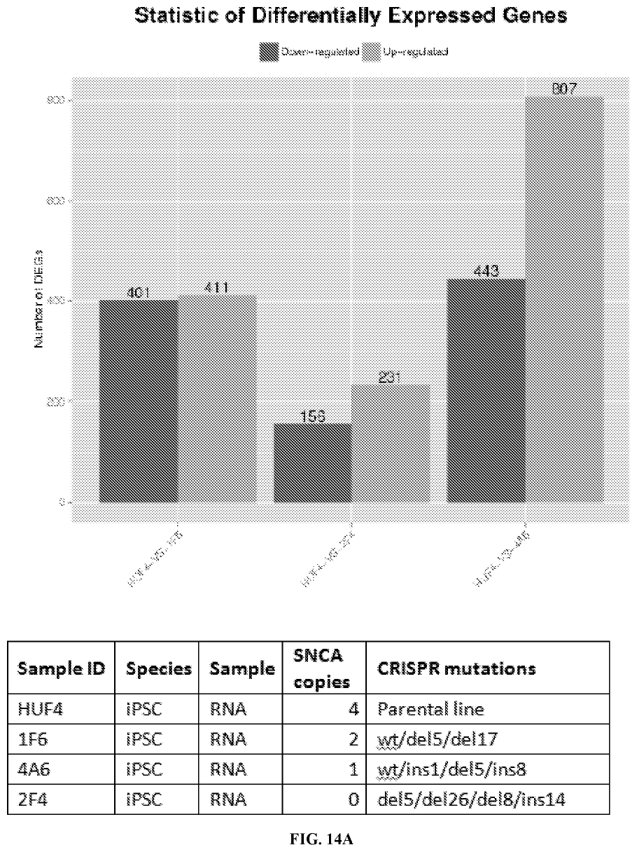

Gene-edited iPSCs are differentiated into neurons using a direct differentiation protocol as shown in FIG. 7 or FIG. 8, allowing for direct differentiation of iPSCs into dopaminergic (DA) neurons without embryoid body formation. This protocol allows for a faster and less labor intensive differentiation of iPSCs into neurons, while still preserving the neuronal precursor cell (NPC) stage of development. At least three clones of each genotype were differentiated for later analyses.

PSC midbrain dopaminergic (DA) neuron differentiation: Differentiation from iPSCs to DA neurons were achieved using PSC midbrain DA differentiation kit (ThermoFisher, A3147701). iPSCs were cultured for 10 days with specification media to generate floor plate progenitors (FPps). FPps were expanded for 10 more days with expansion media to be either cryopreserved or further maturation. The last 15 days of the differentiation process, FPps were developed into functional DA neurons with maturation media. During differentiation, neurons are monitored for viability.

### Example 4

### Further Testing of the iPSC-Differentiated Neurons with Different Functional Gene Copies of the SNCA Gene

Successfully differentiated neuronal lines are analyzed using the following work-flow to maximize utilization of the generated neurons:

Live cell imaging: Morphological or developmental differences (such as the number of generated neurons/genotype, neurite outgrowth) in gene-edited neuronal lines are assessed. As abnormal α-syn levels have been associated with altered transport and interaction of organelles, neurons are transduced with baculoviral vectors encoding fluorescent proteins to investigate protein biosynthesis capability and membrane transport in these live neurons by imaging. Time-resolved expression of organelle-specific targeted fluorescent proteins (Living Colors™) in the nucleus, lysosomes, peroxisomes and mitochondria using Nikon T1 automated microscope is monitored and compared with environmental controls for time resolved microscopy.

Immunocytochemistry: Neuronal immunocytochemistry panel is employed on the engineered neurons for early and mature neuronal markers as well as dopaminergic neurons (Nestin, B3 tubulin, FOXA2, LMX1A, tyrosine hydroxylase). Additionally, these cells are stained for a-syn, 14-3-3 proteins and LRRK2. These stains are conducted on the fluorescent protein-transduced cell lines, thus gaining additional information about co-localization of the antibody targets with cellular organelles.

Cells were fixed at day10 and day35 in 4% PFA and permeabilized with 0.3% triton X-100 in PBS for 5 minutes (except cells stained with tyrosine hydroxylase (TH, Millipore, ab152) and β-III-Tubulin (TUJ1, Covance, MMS-435P)) antibodies, blocked with 10% goat serum for 1 hour at RT, and incubated with primary antibodies (at day10, with FOXA2 (ThermoFisher, A29515) and NESTIN (Milipore, MAB5326)) for overnight at 4C. Indirect immunofluorescence staining was performed with Alexa fluor 488 and 555 conjugated H+L antibodies. Fluorescent images were captured on an Nikon Eclipse Ti inverted fluorescence microscope and analyzed with ANDOR Zyla software.

Taqman gene expression analysis: Total RNA was collected using Qiagen Rneasy Minikit from Trizol treated cells at day0, day10, and day35. cDNA was synthetized using the iScript™ cDNA Synthesis Kit. Taqman probes FAM-MGB labeled SNCA, FAM-MGB labeled TH and for normalization VIC-MGB_PL labeled ACTB were used for relative expression analysis. Relative expression levels were calculated with subsequent ΔCT values that were analyzed using CFX software.

Biochemical assays and immunoblotting: Lysates are prepared from these engineered neurons for biochemical assays and immunoblotting. Function of mitochondrial respiratory chain complex I has been repeatedly shown to be affected in PD. For biochemical analysis, mitochondrial respiratory chain complexes I and IV is immune-precipitated from neuronal cell lysates and analyzed by microplate ELISA for content and activity of these two complexes. If biological material available for biochemistry from differentiated neurons is limiting, these assays are alternatively performed on the intermediate-stage NPCs, as some of the phenotypical and pathological changes observed in SNCA triplication neurons may be observed at the NPC stage.

Western analysis: Neuronal cell lysates are used for Western analysis of selected nuclear and mitochondria-encoded proteins of complex I to assess possible assembly or chaperone defects. Additional Western analysis is focused on mechanisms connecting expression levels as well as phosphorylation status of α-syn and LRRK2, as they have been suggested to play a role in aggregation and toxicity of a-syn. The differential phosphorylation of α-syn and LRRK2 may hint on the functional relationship between LRKK2 and a-syn. Protein content of α-syn interacting proteins, (such as 14-3-3 chaperone proteins17, 18 and the α-syn interacting Polo-Like Kinase 2, PLK219) is investigated. Finally, distribution and amount of the mitochondrial protein import complex TOM40, which is affected by overexpression of a-syn, is assessed.

In case of subtle and non-significant differences in the functional assays comparing single copy differences of the SNCA gene in engineered neurons, these neurons are challenged with neurotoxins that have been shown to contribute to development of PD pathology. Treatment strategies for iPSC derived neurons with both the dopaminergic neurotoxin MPTP and the mitochondrial complex I specific toxin rotenone have been established. For both of these toxins alteration in cellular function in SNCA triplication NPCs is seen.

### Example 5

### Characterization of Isogenic iPSC Lines with Different Functional Copy Numbers of the SNCA Gene via Transcriptome Sequencing

RNA sequencing: Total RNA extraction and DNase I treatment was performed on the isogenic iPSC lines, after which magnetic beads with Oligo (dT) were used to isolate mRNA. Mixed with the fragmentation buffer, the mRNA was fragmented into short fragments. Then cDNA was synthesized using the mRNA fragments as templates. Short fragments were purified and resolved with EB buffer for end reparation and single nucleotide A (adenine) addition. After that, the short fragments were connected with adapters. After agarose gel electrophoresis, the suitable fragments were selected for the PCR amplification as templates. During the QC steps, Agilent 2100 Bioanaylzer and ABI StepOnePlus Real-Time PCR System were used in quantification and qualification of the sample library. Lastly, the library was sequenced using Illumina HiSeq™ 2000 or other sequencer when necessary.

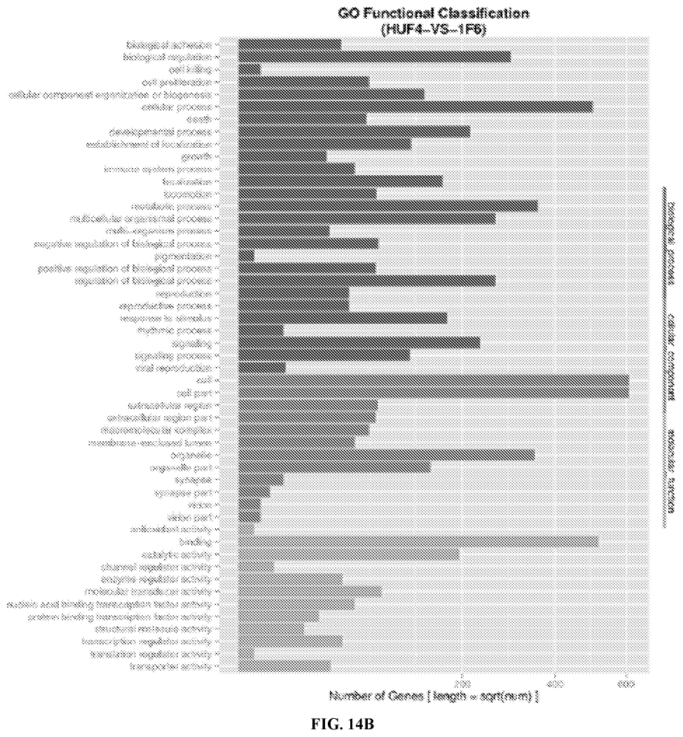

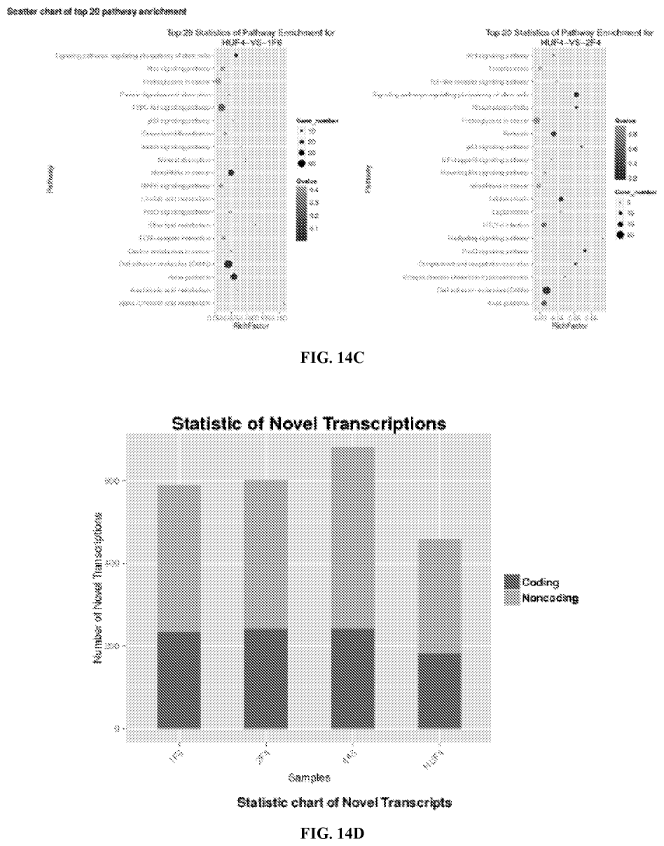

Bioinformatics analysis: Primary sequencing data produced by Illumina HiSeq™ 2000, called as raw reads, were subjected to quality control (QC) that determine if a resequencing step is needed. After QC, raw reads were filtered into clean reads which are aligned to the reference sequences. QC of alignment was performed to determine if resequencing is needed. The alignment data was utilized to calculate distribution of reads on reference genes and mapping ratio. When alignment result passed QC, downstream analysis including gene and isoform expression, deep analysis based on gene expression (PCA/correlation/screening differentially expressed genes and so on), exon expression, gene structure refinement, alternative splicing, novel transcript prediction and annotation, SNP detection, Indel detection, gene fusion was performed. Further, we also can perform deep analysis based on DEGs, including Gene Ontology (GO) enrichment analysis, Pathway enrichment analysis, cluster analysis, protein-protein interaction network analysis and finding transcriptor factor was also performed. FIG. 14A-FIG. 14D exemplify the results obtained.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An isogenic induced pluripotent cell line produced from induced pluripotent stem cell with alpha-synuclein (SNCA) gene triplication, produced by:
(a) contacting the stem cell with SNCA gene triplication with (i) a synthetic polynucleotide that targets a target sequence in one or more of the SNCA genes, and (ii) a genetically engineered vector comprising a gene which encodes a nucleic acid-guided nuclease; and
(b) assessing the cell for copies of the SNCA gene.

2. The cell line of claim 1, wherein the induced pluripotent stem cell with alpha-synuclein (SNCA) gene triplication is human-derived.

3. The cell line of claim 1, wherein one or more of the SNCA genes is a functional SNCA gene.

4. The cell line of claim 1, wherein the synthetic polynucleotide is a guide nucleic acid.

5. The cell line of claim 4, wherein the guide nucleic acid is a guide RNA (gRNA).

6. The cell line of claim 1, wherein the target sequence is in exon 2 of the one or more SNCA genes.

7. The cell line of claim 1, wherein the target sequence is in exon 3 of the one or more SNCA genes.

8. The cell line of claim 1, wherein the target sequence is in exon 4 of the one or more SNCA genes.

9. The cell line of claim 1, wherein the target sequence is in exon 5 of the one or more SNCA genes.

10. The cell line of claim 1, wherein the target sequence comprises: 5′ GAGAAAACCAAACAGGGTG 3′, 5′ GGACTTTCAAAGGCCAAGG 3′, 5′ GCTGCTGAGAAAACCAAAC 3′, 5′ GCTTCTGCCACACCCTGTT 3′, or 5′ GCAGCCACAACTCCCTCCT 3′.

11. The cell line of claim 1, wherein the nuclease introduces a double strand break in the target sequence in one or more the SNCA genes.

12. The cell line of claim 1, wherein the target sequence in the one or more SNCA genes is modified by non-homologous end joining.

13. The cell line of claim 1, wherein the nucleic acid-guided nuclease is a CRISPR nuclease.

14. The cell line of claim 13, wherein the CRISPR nuclease is Cas9.

15. The cell line of claim 1, wherein the cell has three copies of a functional SNCA gene.

16. The cell line of claim 1, wherein the cell has two copies of a functional SNCA gene.

17. The cell line of claim 1, wherein the cell has one copy of a functional SNCA gene.

18. The cell line of claim 1, wherein the cell has zero copies of a functional SNCA gene.

19. The cell line of claim 1, wherein the cell has normal karyotype.

20. The cell line of claim 1, wherein the cell maintains expression of pluripotency markers.

21. The cell line of claim 1, wherein the cell maintains differentiation potential.

22. The cell line of claim 1, wherein SNCA mRNA expression in the cell is proportional to the copies of functional SNCA genes in the cell.

23. The cell line of claim 1, wherein the cell is present in a cell culture.

24. An isogenic induced pluripotent cell line comprising three copies of alpha-synuclein (SNCA) gene, wherein the cell line is derived from induced pluripotent stem cell with alpha-synuclein (SNCA) gene triplication.

25. The cell line of claim 24, wherein the induced pluripotent stem cell with alpha-synuclein (SNCA) gene triplication is human-derived.

26. The cell line of claim 24, wherein the SNCA gene is functional SNCA gene.

27. The cell line of claim 24, wherein the cell has normal karyotype.

28. The cell line of claim 24, wherein the cell maintains expression of pluripotency markers.

29. The cell line of claim 24, wherein the cell maintains differentiation potential.

30. The cell line of claim 24, wherein SNCA mRNA expression in the cell is increased compared to SNCA mRNA expression in a control cell.

31. The cell line of claim 24, wherein SNCA mRNA expression in the cell is comparable to SNCA mRNA expression in a control cell.

32. The cell line of any one of claims 30-31, wherein the control cell comprises two copies of wild-type SNCA gene.

33. The cell line of claim 24, wherein the cell is present in a cell culture.

34. An isogenic induced pluripotent cell line comprising two copies of alpha-synuclein (SNCA) gene, wherein the cell line is produced from induced pluripotent stem cell with alpha-synuclein (SNCA) gene triplication.

35. The cell line of claim 34, wherein the induced pluripotent stem cell with alpha-synuclein (SNCA) gene triplication is human-derived.

36. The cell line of claim 34, wherein the SNCA gene is functional SNCA gene.

37. The cell line of claim 34, wherein the cell has normal karyotype.

38. The cell line of claim 34, wherein the cell maintains expression of pluripotency markers.

39. The cell line of claim 34, wherein the cell maintains differentiation potential.

40. The cell line of claim 34, wherein SNCA mRNA expression in the cell is increased compared to SNCA mRNA expression in a control cell.

41. The cell line of claim 34, wherein SNCA mRNA expression in the cell is comparable to SNCA mRNA expression in a control cell.

42. The cell line of any one of claims 40-41, wherein the control cell comprises two copies of wild-type SNCA gene.

43. The cell line of claim 34, wherein the cell is present in a cell culture.

44. An isogenic induced pluripotent cell line comprising one copy of alpha-synuclein (SNCA) gene, wherein the cell line is produced from induced pluripotent stem cell with alpha-synuclein (SNCA) gene triplication.

45. The cell line of claim 44, wherein the induced pluripotent stem cell with alpha-synuclein (SNCA) gene triplication is human-derived.

46. The cell line of claim 44, wherein the SNCA gene is functional SNCA gene.

47. The cell line of claim 44, wherein the cell has normal karyotype.

48. The cell line of claim 44, wherein the cell maintains expression of pluripotency markers.

49. The cell line of claim 44, wherein the cell maintains differentiation potential.

50. The cell line of claim 44, wherein SNCA mRNA expression in the cell is comparable to SNCA mRNA expression in a control cell.

51. The cell line of claim 44, wherein SNCA mRNA expression in the cell is decreased compared to SNCA mRNA expression in a control cell.

52. The cell line of any one of claims 50-51, wherein the control cell comprises two copies of wild-type SNCA gene.

53. The cell line of claim 44, wherein the cell is present in a cell culture.

54. An isogenic induced pluripotent cell line comprising zero copies of alpha-synuclein (SNCA) gene, wherein the cell line is produced from induced pluripotent stem cell with alpha-synuclein (SNCA) gene triplication.

55. The cell line of claim 54, wherein the induced pluripotent stem cell with alpha-synuclein (SNCA) triplication is human-derived.

56. The cell line of claim 54, wherein the zero copies are zero functional copies of SNCA gene.

57. The cell line of claim 54, wherein the cell has normal karyotype.

58. The cell line of claim 54, wherein the cell maintains expression of pluripotency markers.

59. The cell line of claim 54, wherein the cell maintains differentiation potential.

60. The cell line of claim 54, wherein the cell has reduced differentiation potential compared to a control cell.

61. The cell line of claim 54, wherein SNCA mRNA expression in the cell is decreased compared to SNCA mRNA expression in a control cell.

62. The cell line of any one of claims 60-61, wherein the control cell comprises two copies of wild-type SNCA gene.

63. The cell line of claim 54, wherein the cell is present in a cell culture.

64. A neuronal precursor cell line derived from an isogenic induced pluripotent cell line of any one of the preceding claims.

65. A neuronal cell line derived from an isogenic induced pluripotent cell line of any one of the preceding claims.

66. The neuronal cell line of claim 65, wherein the cell is dopaminergic (DA) neuron.
